(19) United States
(12) Patent Application Publication
(10) Pub. No.: US 2023/0144557 A1
(43) Pub. Date: May 11, 2023

(54) METHOD OF LONG-TERM PRESERVATION OF CHEMICAL AND BIOLOGICAL SPECIES USING SUGAR GLASSES

(71) Applicant: McMaster University, Hamilton (CA)

(72) Inventors: Vincent Ho Yin Leung, Mississauga (CA); Sana Jahanshahi-Anbuhi, Quebec (CA); Carlos Filipe, Ancaster (CA); M. Monsur Ali, Hamilton (CA)

(21) Appl. No.: 17/918,377

(22) PCT Filed: Apr. 13, 2021
(86) PCT No.: PCT/CA2021/050494

§ 371 Date: Oct. 12, 2022

Related U.S. Application Data
(60) Provisional application No. 63/009,041, filed on Apr. 13, 2020.

(51) Int. Cl. A01N 1/02; C12N 7/00; A61K 47/36; A61K 47/26

(52) U.S. Cl. CPC A01N 1/0231; C12N 7/00; A61K 47/36; A61K 47/26; C12N 2795/00032

(57) ABSTRACT

A method of preserving one or more chemical and/or biological species in a polymer matrix comprising pullulan and trehalose is described. The method includes combining the one or more chemical and/or biological species, an aqueous pullulan and a trehalose solution and drying the resultant mixture to provide a solid polymeric matrix in the form of a powder and/or with a water content of less than 10 wt %. The polymeric matrix comprising the one or more chemical and/or biological species and its use, for example, in biological preparations and medicaments is also described.

### CROSS-REFERENCE TO RELATED APPLICATIONS

The present application claims the benefit of priority from U.S. provisional patent application Ser. No. 63/009,041, filed on Apr. 13, 2021, the contents of which are incorporated by reference in their entirety.

### FIELD

The present application relates to the preservation and/or stabilization of chemical and/or biological species in sugar glasses, in particular materials comprising pullulan and trehalose. The application includes the materials and methods for their use in stabilizing and/or preserving chemical and/or biological species.

### BACKGROUND

Bacteriophages (phages) are viruses that infect bacteria. From the early days of their discovery in 1917, lytic bacteriophages have been used as potent antimicrobial agents.^{1,2} An advantage of phage over other antimicrobial agents is its specificity. Whereas most broad-spectrum antimicrobial agents function like a sledgehammer, wiping out any and all bacteria, bacteriophages specifically target only certain species/strains of bacteria within a mixed population. This specificity has made bacteriophage antimicrobials very attractive for food processing/packaging applications, amongst others.³¹⁷⁷ The odor, taste, and texture of most food products, particularly fresh produce, is negatively affected by commercial antimicrobial agents. Bacteriophages exist naturally on fruits and vegetables and adding phage antimicrobials will not affect the appearance, taste, odor, or texture of produce.¹² Bacteriophage antimicrobials are also particularly useful for decontaminating products such as cheese for which the quality of the product strongly depends on the presence of beneficial bacteria, or honey which is consumed raw and without additives or antibiotics. In addition to their specificity, phage antimicrobials have garnered significant attention in the past 20 years as a result of the ever-growing crisis of antibiotic resistance.¹³⁻¹⁶ Presence of multidrug resistant bacteria in animal products poses a serious threat to public health, especially in countries where antibiotics are used liberally and without constraint as an integral part of animal husbandry.^{17,18}

Using phage-impregnated coatings on food preparation surfaces, surfaces in food processing plants, and for food packaging may be a promising way to ensure food safety.^{5,8,14,9} However, to effectively incorporate phage as part of a functional antimicrobial coating, certain challenges must be addressed, including the issue of phage stability.⁷ Bacteriophages are generally resilient to most environmental conditions such as temperature, pH, and salt concentration, although their sensitivity varies significantly amongst strains.²⁰ However, desiccation cannot be endured by many phages and thus a challenge in developing phage-functionalized coatings is finding methods to protect phage against the effects of desiccation.

Amongst methods proposed to date for long-term stabilization of bacteriophage preparations, freeze-drying^{21,22} (also known as lyophilization) and freezing in liquid nitrogen are the most prevalent. Neither method can preserve phage stability unless a protectant, such as glycerol, alginate,^{8,23-28} pectin,^{24,29} chitosan,^{25,26} whey protein,^{16,30} liposome,^{31,32} poly(ethylene oxide)/cellulose diacetate,³³ and sucrose/trehalose³⁴ is present. Also, the lyophilized samples must be maintained in vacuum ampoules for effective phage stabilization. Both methods require access to specialized equipment for sample preparation (freeze dryer, vacuum pumps) and sample storage (liquid nitrogen storage).

Vaccines are an essential part of global health. Every year, millions of lives are saved through vaccination. Unfortunately, almost all available vaccines are thermally labile and must be kept between 2-8° C. at all times to retain their efficacy.³⁵ Failure to maintain an uninterrupted refrigerated supply chain from production to dispensation, called the “cold chain,” leads to vaccine wastage and administration of ineffective vaccines.³⁶ The need for refrigeration is one of the major causes of under-vaccination globally as the cold chain presents economical and logistical problems for vaccination programs. The problem is especially serious for developing countries and remote areas where there are often a lack of dependable cold chain infrastructure and access to reliable electricity is limited.³⁷⁻³⁹

The development of thermally-stable vaccines that can remain active outside of the cold chain can greatly increase the accessibility of vaccination programs and significantly decrease the cost. Therefore, significant efforts have been made in creating thermally-stable vaccines and/or vaccine carriers. One approach has been to engineer vaccines that are thermally stable without preservative adjuvants. The engineering of protein-based vaccine had shown some promise.⁴⁰⁻⁴³ Other reports modified viral vectors to create thermally stable viral vaccines.⁴⁴⁻⁴⁵ Although designing thermally stable vaccines hold some promise, many of the engineered vaccine still have short shelf-life (˜7 days) at elevated temperature (>37° C.). Moreover, engineering new vaccines is labor intensive and the new vaccines must obtain governmental approval before deployment.

Another common approach to thermally stabilize vaccine is the addition of stabilizing adjuvants⁴⁶ In addition to stabilizing adjuvants, vaccines are often dried to further increase thermal stability. Prausntz's group encapsulated inactivated influenza vaccine in microneedle patches with different stabilizing adjuvant formulations and the vaccine maintained immunogenicity after 4 months at 60° C.⁴⁷⁻⁴⁹ Lyophilized anthrax vaccine was found to have preserved immunogenicity after 16 weeks at 40° C.⁵⁰ and lyophilized recombinant ricin toxin A vaccine was stable after 4 weeks at 40° C.⁵¹ Recombinant hepatitis B vaccine and a protein-polysacharide conjugate vaccine for meningitis A was shown to be stable for 24 months at 37° C. after spray drying.⁵² Foam drying of attenuated Salmonella enterica vaccine using trehalose methionine and gelatin were able to maintain vaccine potency for 12 weeks at 37° C.⁵³ Spray drying formulations using sugars and proteins with live attenuated measles vaccine were shown to be stable for up to 8 weeks at 37° C.⁵⁴ Lovalenti et al. stabilized live-attenuated influenza vaccines in a sucrose containing excipient using three drying methods, freeze drying, spray drying, and foam drying. It was found that foam drying with the right excipient composition produced the most thermally stable vaccine that had a shelf life of 4.5 months at 37° C.⁵⁵ Different lyophilized formulations of rotavirus vaccines were able to retain potency for 20 months at 37° C.⁵⁶ for up to 20 months.⁵⁷. Alcock et al. used sucrose and trehalose to dry adenovirus and modified vaccinia virus Ankara onto polypropylene or glass fiber membranes, the viruses retained titer for up to 6 months at 45° C.⁵⁸ Many of the reports use freeze drying, spray drying, or foam drying which all require specialized equipment for sample preparation (freeze dryer, vacuum pumps) and expose vaccines to extreme temperatures or pressure conditions.⁵³ Moreover, some formulations require a large number of adjuvants which can increase the cost and complexity of the vaccine product.

Pullulan is a polysaccharide that has excellent film forming properties and has been used in the food industry as an oxygen barrier to prolong the shelf life of foods.⁵⁹⁻⁶⁵ Previous studies have shown that pullulan is able to provide outstanding thermal stability and protection against oxidation of various labile biomolecules.⁶⁶⁻⁶⁷ Trehalose is a disaccharide sugar that has been used extensively as a cryoprotectant and stabilizing agent during lyophilization.⁶⁸⁻⁷⁹

U.S. Pat. No. 7,749,538 describes a shaped product such as a film, sheet or film capsule shape having a high pullulan content in combination with trehalose wherein the content of the pullulan provides a shaped product that has stability to changes in humidity.

US20190111006 describes a solid material comprising pullulan and trehalose (optionally called a “PT material”) in the form of a film, coating or shaped object, that possesses a synergistic effect that leads to long-term stability of chemical and/or biological species. The contents of this application are specifically incorporated herein by reference in their entirety.

### SUMMARY

The Applicant has found that the combination of pullulan and trehalose in a solid material (optionally called a “PT material”), has beneficial properties when in the form of a powder. Further, the Applicant has found that PT material dried to, and maintained at, a water/moisture content of 10 wt % or less, has beneficial properties for storage and transportation, in particular for degradation-sensitive medicaments.

Accordingly, the present application includes a polymer matrix comprising pullulan and trehalose and one or more chemical and/or biological species, wherein the one or more chemical and/or biological species are incorporated within the polymer matrix and the polymer matrix preserves and/or stabilizes the one or more chemical and/or biological species and the polymer matrix is in powder form.

The present application includes a polymer matrix comprising pullulan and trehalose and one or more chemical and/or biological species, wherein the one or more chemical and/or biological species are incorporated within the polymer matrix and the polymer matrix preserves and/or stabilizes the one or more chemical and/or biological species and the polymer matrix has a water content of 10 wt % or less.

The present application also includes a method of preserving and/or stabilizing one or more chemical and/or biological species comprising:
a) combining the one or more chemical and/or biological species, an aqueous solution comprising pullulan and an aqueous solution comprising trehalose to provide a mixture;
b) drying the mixture to form a polymer matrix which preserves and/or stabilizes the one or more chemical and/or biological species; and
c) converting the polymer matrix into powder form.

The present application also includes a method of preserving and/or stabilizing one or more chemical and/or biological species comprising:
a) combining the one or more chemical and/or biological species, an aqueous solution comprising pullulan and an aqueous solution comprising trehalose to provide a mixture; and
b) drying the mixture to form a polymer matrix which preserves and/or stabilizes the one or more chemical and/or biological species;
wherein the polymer matrix is dried to a water content of less than 10 wt % and is optionally converted to powder form.

The present application also includes a method of creating a material for preserving chemical and/or biological species comprising:
a) combining one or more chemical and/or biological species with an aqueous pullulan and trehalose solution to provide the chemical and/or biological species in solution;
b) drying the solution to provide a solid polymeric matrix; and
c) converting the matrix into powder form.

The present application also includes a method of creating a material for preserving chemical and/or biological species comprising:
a) combining one or more chemical and/or biological species with an aqueous pullulan and trehalose solution to provide the chemical and/or biological species in solution; and
b) drying the solution to provide a solid polymeric matrix;
wherein the solid polymer matrix is dried to a water content of less than 10 wt % and is optionally converted to powder form.

In some embodiments, at least one of the species is a biomolecule. In some embodiments, the biomolecule is selected from one or more of protein, enzyme, antibody, peptide, nucleic acid, phage, antidote, antigen and vaccine.

In some embodiments, at least one of the species a microorganism. In some embodiments, wherein the microorganism is selected from one or more of anaerobic bacteria, aerobic bacteria, mammalian cells, bacterial cells and viruses.

The present application also includes a pullulan/trehalose powder comprising one or more chemical and/or biological species wherein the wt % pullulan in the dried pullulan/trehalose powder is less than 50 wt %.

The present application also includes a dried pullulan/trehalose powder comprising one or more chemical and/or biological species wherein the wt % pullulan in the dried pullulan/trehalose powder is in the range of 30-45 wt %.

The present application also includes a PT material comprising one or more chemical and/or biological species in the form of a powder, and a method of making a PT material comprising one or more chemical and/or biological species in the form of a powder. In some embodiments, a dried PT material comprising one or more chemical and/or biological species is formed and then mechanically converted into a powder. In some embodiments, the initial form (i.e. the form converted into a powder) is a film. In some embodiments, a mixture comprising a pullulan/trehalose solution and one or more chemical and/or biological species is dried by vacuum drying. In some embodiments, the dried PT material comprises less than 10%, less than 9%, less than 8%, less than 7%, less than 6% or less than 5% water by weight.

The present application also includes methods of making, storing and/or transporting a PT material comprising one or more chemical and/or biological species. In some embodiments, a PT material comprising one or more chemical and/or biological species is dried and/or sealed into a package under a vacuum or in an environment with less than 50%, less than 45, less than 40, less than 35, or less than 33%, relative humidity. In some embodiments, the packaged PT material comprising the one or more chemical and/or biological species has a moisture (i.e. water) content of 10 wt % or less. In some embodiments, the packaged PT material comprising the one or more chemical and/or biological species is stored and/or transported at a temperature in the range of −20° C. to 40° C. In some embodiments, the PT material comprising one or more chemical and/or biological species is in powder form.

The present application also includes methods of reconstituting and/or administering a medicament comprising pullulan/trehalose and one or more chemical and/or biological species, for example an antigen. In some embodiments, a PT material comprising one or more chemical and/or biological species is provided in the form of a powder in a sealed container delivered to the administration site or place of treatment. In some embodiments, the PT material has a water content of 10 wt % or less. In some embodiments, a diluent is mixed with the PT material at the administration site/place of treatment to dissolve the PT material and produce an aqueous medicament, for example an aqueous vaccine. In some embodiments, the diluent is added to a container containing the PT material, the PT material is added to a container of the diluent, the PT material and the diluent are transferred to another container, or a barrier between the PT material and the diluent in a container is broken. In some embodiments, the diluent contains water, for example sterile water, and optionally other compounds such as an adjuvant, a salt, a buffer, etc. The aqueous medicament is then administered to a patient, for example by way of injection (for example intramuscular or subcutaneous injection), ingestion (oral administration) or nasal spray (intranasal administration).

The present application also includes a PT material comprising one or more chemical and/or biological species for use in a medicament, for example an immunogenic composition, and/or for use in providing a therapy, for example an immune response, in a patient. The present application also describes a method of providing a therapy, such as an immune response, in a patient comprising administering an effective dose of a medicament comprising one or more chemical and/or biological species, for example an antigen, to the patient, wherein the one or more chemical and/or biological species was previously combined with the PT material.

The present application also describes a method of making a stabilized composition, for example an immunogenic composition, comprising one or more chemical and/or biological species, for example an antigen. In some embodiments, the method includes mixing one or more chemical and/or biological species with a pullulan/trehalose solution, drying the mixture, and converting the dried mixture to a powder. In addition, or alternatively, the method includes mixing one or more chemical and/or biological species with a pullulan/trehalose solution, and drying the mixture to a water content of less than 10 wt %.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

The embodiments of the application will now be described in greater detail with reference to the attached drawings in which:

FIG. 1 shows a kit and process for administering a medicament to a patient in an embodiment of the present application.

FIG. 2 shows absorbance over time of a dye released from complete and crushed (powdered) samples of PT material after adding water to dissolve the PT material.

FIG. 3 shows the effect of storage temperature for a period of four days on phage stored in an exemplary PT material of the application.

FIG. 4 shows the residual water content in films created using two different exemplary initial volumes of PT solution as a function of drying time under vacuum.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise. For example, an embodiment including “a bacteriophage” or “a vaccine” should be understood to present certain aspects with one bacteriophage or vaccine, or two or more additional (and different) bacteriophage or vaccines.

The term “and/or” as used herein means that the listed items are present, or used, individually or in combination. In effect, this term means that “at least one of” or “one or more” of the listed items is used or present.

The term “biomolecule” as used herein means an organic macromolecule (such as a protein or nucleic acid) in living organisms.

The term “microorganism” as used herein means a microscopic organism that may exist as a single cell or as a colony of cells

The term “preserving” or “preservation” as used herein with respect to the chemical and/or biological species means to maintain at least a measurable or detectable level of function or activity for the chemical and/or biological species for a desired period of time under specified conditions.

The term “stabilizing” or “stabilization” as used herein with respect to the chemical and/or biological species refers to any reduction in the degradation or loss of activity of the chemical and/or biological species compared to a control.

The term “drying” as used herein refers to a process of allowing a solution of a polymer to cure or set, by removal of water/moisture, until a solid, movable material is obtained.

The term “pullulan” as used herein refers to a natural polysaccharide which is produced extracellularly by Aurebasidium pullulans when cultivated with starch hydrolyzates as a carbon source.

The term “trehalose” as used herein refers to (D)-(+)-trehalose which is a disaccharide composed of two glucose molecules bound together via the α,α-1,1-glucosidic linkage.

The expression “incorporated within” as used herein means that the one or more biological and/or chemical species are interspersed throughout the pullulan/trehelose polymer matrix.

The term “polymer matrix” as used herein means a material which is made of at least one polymer and which forms a surrounding medium or structure.

The term “food grade” as used herein means that the specified material is compatible for ingestion by humans and/or animals.

The term “medical grade” as used herein means that the specified material is compatible for administration to humans and/or animals including, for example, by way of injection.

The term “vaccine” as used herein may mean, where appropriate given the context, an antigen of a vaccine, but does not necessarily exclude the presence of other parts of a vaccine, such as an adjuvant or diluent.

The term “immunogenic” as used herein relates to or denotes substances able to produce an immune response.

The term “essentially free from” as used herein means that the presence of the stated features, elements, or components, is in an amount that does not materially affect the characteristics of the composition or material being referenced.

As used herein, the term “effective amount” or “therapeutically effective amount” means an amount that is effective, at dosages and for periods of time necessary to achieve a desired result.

The term “medicament” as used herein refers to any substance used for medical treatment or therapy.

### II. Compositions of the Application

The Applicant has found that a material made from pullulan and trehalose acts synergistically to provide a stabilized matrix that is capable of preserving and/or stabilizing chemical and/or biological species, such as bacteriophages and viruses, that are incorporated within the matrix and further certain beneficial properties are provided when the matrix is in powder form. For example, the powdered polymer matrixes of the present application have faster dissolution rates. The Applicant has also found that PT material dried to, and maintained at, a water content of 10 wt % or less, has beneficial properties for storage and transportation, in particular for degradation-sensitive medicaments.

Accordingly, the present application includes a polymer matrix comprising pullulan and trehalose and one or more chemical and/or biological species, wherein the one or more chemical and/or biological species are incorporated within the polymer matrix and the polymer matrix preserves and/or stabilizes the one or more chemical and/or biological species, wherein the polymer matrix is in the form of a powder.

The present application includes a polymer matrix comprising pullulan and trehalose and one or more chemical and/or biological species, wherein the one or more chemical and/or biological species are incorporated within the polymer matrix and the polymer matrix preserves and/or stabilizes the one or more chemical and/or biological species and the polymer matrix has a water content of 10 wt % or less.

In some embodiments the polymer matrix comprises about 10 wt % to about 50 wt % of pullulan and about 50 wt % to about 90 wt % of trehalose, based on the dry weight of the matrix. In some embodiments the polymer matrix comprises about 20 wt % to about 40 wt % of pullulan and about 60 wt % to about 80 wt % of trehalose, based on the dry weight of the matrix. In some embodiments the polymer matrix comprises about 30 wt % to about 40 wt % of pullulan and about 60 wt % to about 70 wt % of trehalose, based on the dry weight of the matrix. In some embodiments the polymer matrix comprises about 37 wt % of pullulan and about 63 wt % of trehalose, based on the dry weight of the matrix.

In some embodiments the pullulan has a molecular weight in the range of about 100,000 to about 200,000. Pullulan having such molecular weights is commercially available, for example from Hayashibara Co, Ltd., Okayama, Japan. Trehalose is available from a variety of commercial sources, including, for example, Hayashibara Co, Ltd., Okayama, Japan. In some embodiments, for use in food products, the pullulan and trehalose are both food grade materials. In some embodiments, for use in medical products, the pullulan and trehalose are both medical grade, or pharmaceutically acceptable, materials.

In some embodiments, the polymer matrix is in the form of a powder. In some embodiments, the polymer matrix is formed into a powder by milling, crushing and/or grinding. In some embodiments, the powder comprises particles having a median surface area:volume ratio of 10:1 mm⁻¹ to about 120:1 mm⁻¹. In some embodiments, the powder comprises particles having a median surface area:volume ratio of 10:1 mm⁻¹ to about 100:1 mm⁻¹. In some embodiments, the powder comprises particles having a median surface area:volume ratio of 12:1 mm⁻¹ to about 60:1 mm⁻¹. In some embodiments, the powder comprises particles having a median surface area:volume ratio of 12:1 mm⁻¹ to about 40:1 mm⁻¹.

In some embodiments, the polymer matrix is formed first into a dried film or a shaped object prior to forming into a powder. In some embodiments, the dried film or shaped object is amorphous. In some embodiments, the shaped object is a pill or capsule.

In some embodiments, the polymer matrix has a water content of less than 10 wt %. In some embodiments, the polymer matrix has a water content of less than 9 wt %. In some embodiments, the polymer matrix has a water content of less than 8 wt %. In some embodiments, the polymer matrix has a water content of less than 7 wt %. In some embodiments, the polymer matrix has a water content of less than 6 wt %. In some embodiments, the polymer matrix has a water content of less than 5 wt %. In some embodiments, the polymer matrix has a water content of about 5 wt % to about 10 wt %. In some embodiment, the polymer matrix has a water content of about 6 wt % to about 10 wt %. In some embodiment, the polymer matrix has a water content of about 7 wt % to about 10 wt %. In some embodiment, the polymer matrix has a water content of about 8 wt % to about 10 wt %.

In some embodiments, the polymer matrix powder comprises less than 10%, less than 9%, less than 8%, less than 7%, less than 6% or less 5%, by weight, of water. In some embodiments, the polymer matrix powder comprises less than 10%, less than 9% or less than 8%, by weight, of water.

In some embodiments, the one or more chemical and/or biological species are preserved and/or stabilized without requiring refrigeration. In some embodiments, the one or more chemical and/or or biological species are preserved at a temperature of from about 2° C. to about 40° C., about 10° C. to about 30° C. or about 20° C. to about 25° C. In some embodiments, the one or more chemical and/or or biological species are preserved at a temperature of from about −20° C. to about 40° C., about 10° C. to about 30° C. or about 20° C. to about 25° C. In some embodiments, the one or more chemical and/or biological species are preserved and/or stabilized for at least 3 months at the above temperatures. In some embodiments, the one or more chemical and/or biological species are preserved and/or stabilized for at least 4 days, or from 4 to 10 days at temperatures below freezing.

In some embodiments, the one or more chemical species is a biomolecule. In some embodiments, the biomolecule is chosen from one or more of a protein, an enzyme, an antibody, a peptide, a nucleic acid, an antidote and a vaccine. In some embodiments, the biomolecule is a vaccine. In some embodiments, the biomolecule is an immunogenic protein. In some embodiments, the biomolecule is an antigen. In some embodiments, the one or more biological species is a microorganism. In some embodiments, the microorganism is chosen from one or more of anaerobic bacteria, aerobic bacteria, mammalian cells, bacterial cells and viruses. In some embodiments, the microorganism is a virus. In some embodiments, the virus is a bacteriophage. In some embodiments, the virus is an enveloped virus. In some embodiments, the virus is a DNA virus. In some embodiments, the virus is Herpes Simplex Virus (HSV-2). In some embodiments, the virus is HSV-2 In some embodiments, the virus is a RNA virus. In some embodiments, the virus is influenza virus. In some embodiments, the virus is PR8. In some embodiments, the virus is formulated for administration in a biological preparation. In some embodiments, the virus is formulated for administration as a live-attenuated vaccine. In some embodiments, the virus is formulated for administration as an inactivated vaccine.

In some embodiments the one or more chemical species are biomolecules that act as immunogens or that are used to generate an immune response, including, DNA, RNA, peptides and/or proteins. In some embodiments, these biomolecules are incorporated within the matrix along with other agents that are used in vaccine formulations, such as adjuvants. In some embodiments, the polymer matrix further comprises one or more additional substances or additives such as, but not limited to, seasonings, spices, colorings, flavors, emulsifiers and plasticizers. In some embodiments, the physical properties of the polymer matrix, such as solubility, transparency, tactile impression, texture, plasticity, etc. are changed using additives.

In some embodiments, the polymer matrix is without or essentially free from any additives that are non-GRAS or not acceptable for injection in a person. In some embodiments, the polymer matrix is without or essentially free from PMAL-C16. In some embodiments, the polymer matrix is free from or without, or contains less than 0.1% to 10%, of a zwitterionic surfactant having a lipid group with a chain length of 13-30 carbon atoms. In some embodiments, the polymer matrix is free from or without, or contains less than 0.1 mg/ml to 50 mg/ml, of a zwitterionic surfactant having a lipid group with a chain length of 13-30 carbon atoms.

The present application also includes an immunogenic composition comprising an antigen, the antigen formulated in a polymer matrix of the application.

### III. Methods and Uses of the Application

The Applicants have found that chemical and/or biological species can be preserved and/or stabilized by incorporating the species within in a polymer matrix comprising certain amounts of pullulan and trehalose, wherein the polymer matrix is in powder form and/or wherein the polymer matrix has a water content of less than 10 wt %.

Accordingly, the present application includes a method of preserving and/or stabilizing one or more chemical and/or biological species comprising:
a) combining the one or more chemical and/or biological species, an aqueous solution comprising pullulan and an aqueous solution comprising trehalose to provide a mixture;
b) drying the mixture to form a polymer matrix which preserves and/or stabilizes the one or more chemical and/or biological species; and
c) converting the polymer matrix into powder form.

In some embodiments, the trehalose is added to an aqueous solution of pullulan and the one or more chemical and/or biological species. In some embodiments, the trehalose is added at a concentration of about 0.1 to 1 M, or about 0.5 M.

In some embodiments, the one or more chemical and/or biological species, an aqueous solution comprising pullulan and an aqueous solution comprising trehalose are mixed thoroughly to ensure uniform distribution of all of the ingredients.

In some embodiments, one or more additional substances or additives such as, but not limited to, seasonings, spices, colorings, flavors, emulsifiers, salts, adjuvants, buffers and plasticizers are added to the mixture, and/or to the one or more chemical and/or biological species, the aqueous solution comprising pullulan or the aqueous solution comprising trehalose prior to drying. In some embodiments, the physical properties of the polymer matrix, such as solubility, transparency, tactile impression, texture, plasticity, etc. is changed using additives.

In some embodiments, the mixture is drop cast into a specific shape prior to drying. In some embodiments, the shape is a pill or a capsule.

In some embodiments, the mixture is formed into a film prior to drying. In some embodiments, the mixture is formed into a thin film of any of a variety of shapes, for example a strip or patch. In some embodiments, the shaped object is a pill or capsule.

In some embodiments, the polymer matrix is formed into a powder by milling, crushing and/or grinding. In some embodiments, the powder comprises particles having a median surface area:volume ratio of 10:1 mm⁻¹ to about 120:1 mm⁻¹. In some embodiments, the powder comprises particles having a median surface area:volume ratio of 10:1 mm⁻¹ to about 100:1 mm⁻¹. In some embodiments, the powder comprises particles having a median surface area:volume ratio of 12:1 mm⁻¹ to about 60:1 mm⁻¹. In some embodiments, the powder comprises particles having a median surface area:volume ratio of 12:1 mm⁻¹ to about 40:1 mm⁻¹.

In some embodiments the polymer matrix comprises about 10 wt % to about 50 wt % of pullulan and about 50 wt % to about 90 wt % of trehalose, based on the dry weight of the matrix. In some embodiments the polymer matrix comprises about 20 wt % to about 40 wt % of pullulan and about 60 wt % to about 80 wt % of trehalose, based on the dry weight of the matrix. In some embodiments the polymer matrix comprises about 30 wt % to about 40 wt % of pullulan and about 60 wt % to about 70 wt % of trehalose, based on the dry weight of the matrix. In some embodiments the polymer matrix comprises about 37 wt % of pullulan and about 63 wt % of trehalose, based on the dry weight of the matrix.

In some embodiments the pullulan has a molecular weight in the range of about 100,000 to about 200,000. Pullulan having such molecular weights is commercially available, for example from Hayashibara Co, Ltd., Okayama, Japan. Trehalose is available from a variety of commercial sources, including, for example, Hayashibara Co, Ltd., Okayama, Japan. In some embodiments, for use in food products, the pullulan and trehalose are both food grade materials. In some embodiments, for use in medical products, the pullulan and trehalose are both medical grade, or pharmaceutically acceptable, materials.

In some embodiments, the one or more chemical and/or biological species are preserved and/or stabilized without requiring refrigeration. In some embodiments, one or more chemical or biological species are preserved at a temperature of from about 2° C. to about 40° C., about 10° C. to about 30° C. or about 20° C. to about 25° C. In some embodiments, the one or more chemical and/or or biological species are preserved at a temperature of from about −20° C. to about 40° C., about 10° C. to about 30° C. or about 20° C. to about 25° C. In some embodiments, the one or more chemical and/or biological species are preserved and/or stabilized for at least 3 months at the above temperatures. In some embodiments, the one or more chemical and/or biological species are preserved and/or stabilized for at least 4 days, or from 4 to 10 days at temperatures below freezing.

In some embodiments, the one or more chemical species is a biomolecule. In some embodiments, the biomolecule is chosen from one or more of a protein, an enzyme, an antibody, a peptide, a nucleic acid, an antidote and a vaccine. In some embodiments, the biomolecule is an immunogenic protein. In some embodiments, the biomolecule is an antigen. In some embodiments, the biomolecule is a vaccine. In some embodiments, the one or more biological species is a microorganism. In some embodiments, the microorganism is chosen from one or more of anaerobic bacteria, aerobic bacteria, mammalian cells, bacterial cells and viruses. In some embodiments, the microorganism is a virus. In some embodiments, the virus is a bacteriophage. In some embodiments, the virus is an enveloped virus. In some embodiments, the virus is a DNA virus. In some embodiments, the virus is Herpes Simplex Virus (HSV-2). In some embodiments, the virus is HSV-2 TK⁻. In some embodiments, the virus is a RNA virus. In some embodiments, the virus is influenza virus. In some embodiments, the virus is PR8. In some embodiments, the virus is formulated for administration in a biological preparation. In some embodiments, the virus is formulated for administration as a live-attenuated vaccine. In some embodiments, the virus is formulated for administration as an inactivated vaccine.

In some embodiments the one or more chemical species are biomolecules that act as immunogens or that are used to generate an immune response, including, DNA, RNA, peptides and/or proteins. In some embodiments, these biomolecules are incorporated within the matrix along with other agents that are used in vaccine formulations, such as adjuvants.

In some embodiments, the polymer matrix is without or essentially free from any additives that are non-GRAS or not acceptable for injection in a person. In some embodiments, the polymer matrix is without or essentially free from PMAL-C16. In some embodiments, the polymer matrix is free from or without, or contains less than 0.1% to 10%, of a zwitterionic surfactant having a lipid group with a chain length of 13-30 carbon atoms. In some embodiments, the polymer matrix is free from or without, or contains less than 0.1 mg/ml to 50 mg/ml, of a zwitterionic surfactant having a lipid group with a chain length of 13-30 carbon atoms.

In some embodiments, the present application also includes a method of preserving and/or stabilizing one or more chemical and/or biological species comprising:
a) combining the one or more chemical and/or biological species, an aqueous solution comprising pullulan and an aqueous solution comprising trehalose to provide a mixture; and
b) drying the mixture to form a polymer matrix which preserves and/or stabilizes the one or more chemical and/or biological species;
wherein the polymer matrix is dried to a water content of less than 10 wt %.

In some embodiments an aqueous mixture of pullulan, trehalose and one or more chemical and/or biological species is dried in a partial vacuum, dried under a nitrogen-enhanced atmosphere, and/or dried in a desiccated environment. In some embodiments, the mixture is not freeze dried. Freeze drying can produce PT materials with a cloudy or opaque appearance, which is believed to be caused by the formation of crystals in the PT material. The cloudy or opaque appearance produced during freeze drying is associated with a loss of activity of at least some chemical or biological species intended to be preserved and/or stabilized by the PT material.

In some embodiments, the polymer matrix is dried to a water content of less than 10 wt %. In some embodiments, the polymer matrix is dried to a water content of less than 9 wt %. In some embodiments, the polymer matrix is dried to a water content of less than 8 wt %. In some embodiments, the polymer matrix is dried to a water content of less than 7 wt %. In some embodiments, the polymer matrix is dried to a water content of less than 6 wt %. In some embodiments, the polymer matrix is dried to has a water content of less than 5 wt %. In some embodiments, the polymer matrix is dried to a water content of about 5 wt % to about 10 wt %. In some embodiment, the polymer matrix is dried to a water content of about 6 wt % to about 10 wt %. In some embodiment, the polymer matrix is dried to a water content of about 7 wt % to about 10 wt %. In some embodiment, the polymer matrix is dried to a water content of about 8 wt % to about 10 wt %.

In some embodiments, the PT materials of the present application comprising one or more chemical and/or biological species and dried to a water content of less than 10 wt % are formed in, or has the form of, small particles, for example a powder. In some embodiments, a brittle solid material is formed, which is mechanically broken into a powder. In some embodiments, the powder comprises particles having a median surface area:volume ratio of 10:1 mm⁻¹ to about 120:1 mm⁻¹. In some embodiments, the powder comprises particles having a median surface area:volume ratio of 10:1 mm⁻¹ to about 100:1 mm⁻¹. In some embodiments, the powder comprises particles having a median surface area:volume ratio of 12:1 mm⁻¹ to about 60:1 mm⁻¹. In some embodiments, the powder comprises particles having a median surface area:volume ratio of 12:1 mm⁻¹ to about 40:1 mm⁻¹.

Once solidified, the PT material with one or more chemical and/or biological species may be subjected to freezing. The PT material may be used to preserve and/or stabilize one or more chemical or biological species over a temperature range of at least −20° C. to 40° C.

The PT material with one or more chemical and/or biological species may be stored in a sealed container. Optionally, the sealed container may also contain a desiccant and/or be sealed while under vacuum. However, acceptable performance can be achieved if the PT material with one or more chemical and/or biological species is filled and sealed into containers while inside of a room or other vessel (for example a biosafety or other controlled environment cabinet) having 50% relative humidity or less, or 45%, 40%, 35% or 33% relative humidity or less. The permanent gasses in the room or other vessel may be air or a more nearly inert gas, for example nitrogen enriched air or nitrogen. The gasses in the room or other vessels may be treated, for example filtered or passed through a sorbent, to remove contaminants.

The present application also includes methods of reconstituting and/or administering a medicament comprising pullulan/trehalose and one or more chemical and/or biological species, for example an antigen. In some embodiments, a PT material comprising one or more chemical and/or biological species is provided in the form of a powder in a sealed container delivered to the administration site or place of treatment. In some embodiments, the PT material has a water content of 10 wt % or less. In some embodiments, a diluent is mixed with the PT material at the administration site/place of treatment to dissolve the PT material and produce an aqueous medicament, for example an aqueous vaccine. In some embodiments, the diluent is added to a container containing the PT material, the PT material is added to a container of the diluent, the PT material and the diluent are transferred to another container, or a barrier between the PT material and the diluent in a container is broken. In some embodiments, the diluent contains water, for example sterile water, and optionally other compounds such as an adjuvant, a salt, a buffer, etc. The aqueous medicament is then administered to a patient, for example by way of injection (for example intramuscular or subcutaneous injection), ingestion (oral administration) or nasal spray (intranasal administration).

In some embodiments, the PT material with one or more chemical and/or biological species are stored in a container. The container may be sealed, for example by a septum. In some embodiments, multiple PT materials, each with one or more chemical and/or biological species, are stored in the same container. For example, one PT material may contain one antigen while another PT material contains another antigen, one PT material may contain an antigen while one or more other PT materials contain one or more of a buffer, a salt or an adjuvant. In some embodiments. an aqueous mixture including pullulan, trehalose and one or more chemical and/or biological species is reconstituted by adding a diluent, for example by drawing the diluent into a syringe and then injecting the diluent into the container through the septum. In some embodiments, the container is agitated, for example shaken, to help increase the rate of dissolution of the PT material or to help ensure than no un-dissolved PT material remains. A portion of the reconstituted mixture can then be drawn out of the container by way of a needle inserted through the septum. The reconstituted mixture can then be administered to a patient (i.e. a human or non-human mammal or other animal), for example by being injected into the patient. Alternatively, in some embodiments, the reconstituted mixture is administered to the patient by an intranasal route by way of an atomizer or sprayer, or the patient may swallow some of the reconstituted mixture.

The present application also includes a polymer matrix comprising pullulan and trehalose and one or more chemical and/or biological species, wherein the one or more chemical and/or biological species are incorporated within the polymer matrix and the polymer matrix preserves and/or stabilizes the one or more chemical and/or biological species, and the polymer matrix is in the form of a powder for use as a medicament, for example an immunogenic composition. In some embodiments, the medicament is reconstituted from the powder and injected into a mammal. In some embodiments, the one or more chemical and/or biological species are immunogenic and the polymer matrix is for use in a method of providing an immune response in a mammal.

The present application also includes a method of providing an immune response in a mammal comprising administering, to a mammal in need thereof, an effective amount of a polymer matrix comprising pullulan and trehalose and one or more chemical and/or biological species, wherein the one or more chemical and/or biological species are immunogenic and are incorporated within the polymer matrix and the polymer matrix preserves and/or stabilizes the one or more chemical and/or biological species, and the polymer matrix is in the form of a powder. In some embodiments, a diluent is added to the powder to provide an aqueous mixture and administering the aqueous mixture to the mammal, for example by injecting the aqueous mixture into the mammal. The present application also includes a method of making and/or storing and/or transporting a stabilized medicament or component thereof comprising a) producing a mixture of pullulan, trehalose and one or more chemical and/or biological species, b) drying the mixture to 10 wt % water content or below to provide a dried material and c) packaging the dried material in a sealed container. In some embodiments, the dried material is in the form of a powder. In some embodiments, the container comprises a septum. In some embodiments, the mixture is reconstituted by injecting a diluent through the septum. In some embodiments, the container is stored and/or transported at a temperature between −20° C. and 40° C. In some embodiments, the the container is stored for a period of time above 8° C.

The present application also includes a method of making and/or storing and/or transporting a stabilized medicament or component thereof comprising a) producing a mixture of pullulan, trehalose and one or more chemical and/or biological species, b) drying the mixture to form a polymer matrix, c) forming the polymer matrix into a powder, and d) packaging the dried powder in a sealed container. In some embodiments, the mixture is dried to a water content of less than 10 wt %. In some embodiments, the container comprises a septum. In some embodiments, the mixture is reconstituted by injecting a diluent through the septum. In some embodiments, the container is stored and/or transported at a temperature between −20° C. and 40° C. In some embodiments, the the container is stored for a period of time above 8° C.

In some embodiments, a syringe is pre-loaded with a PT material comprising one or more chemical and/or biological species in the form of a powder and/or having a water content of less than 10 wt %. The PT material dissolves when a diluent is drawn into the syringe. The resulting aqueous mixture can be injected into a patient through a needle attached to the same syringe. In some embodiments, the reconstituted mixture is a whole vaccine (i.e. a combination of antigen and any required adjuvants or other additives).

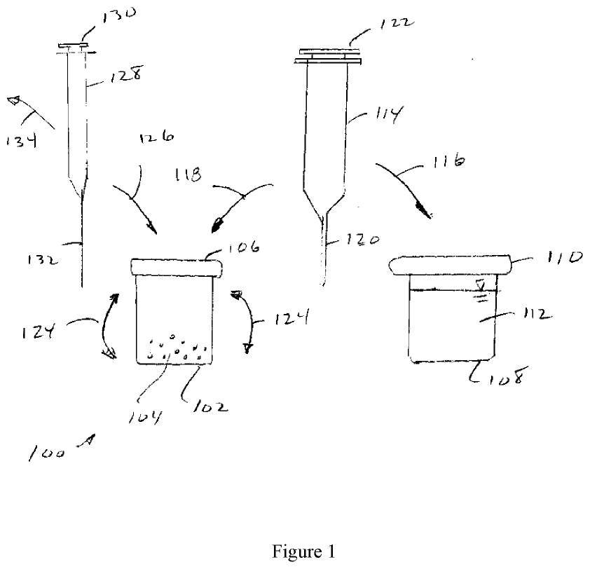

FIG. 1 shows an example of a process or kit 100 for storing, delivering and/or administering a medicament, for example a vaccine. A PT material 104 containing one or more chemical and/or biological species, for example an antigen, is produced in the form of a dry powder in a vial 102. The vial 102 has a cap 106 including an injection septum. A vial 108 is produced containing diluent 112 under a cap 110 including an injection septum. In step 116, a syringe 114 is loaded with some of the sterile water 112. In the example shown, a needle 120 attached to syringe 114 is inserted into the vial 108 through the septum in its cap 110 and the plunger 122 is pulled out to draw up some of the diluent 112 before withdrawing the syringe 114 from the vial 108. In step 118, the diluent 112 is added to the vial 102. In the example shown, the needle 120 of syringe 114 is inserted through the septum in the cap 106. The plunger 122 is pushed into inject some of the diluent 112 into the vial. The injected diluent 112 may cause sufficient mixing to dissolve the PT material 104 thereby releasing the one or more chemical and/or biological species. Optionally, in step 124, the vial 102 is shaken until the PT material 104 is essentially dissolved, for example by inverting the vial 102 a few times or until no particles are visible to the eye. The vial 102 now contains reconstituted medicament, including dissolved PT material and the one or more chemical and/or biological species. In step 126 some, for example an effective amount, i.e. a single dose or one dose according to a multi-dose regimen, of the reconstituted medicament is loaded in a syringe 128. A needle 132 attached to the syringe 128 is inserted through the septum in cap 106 and plunger 130 pulled back to draw some of the reconstituted medicament into the syringe 128. Syringe 128 is withdrawn from the vial 102. In step 134, the reconstituted medicament is injected into a patient from syringe 128, for example by way of intramuscular or subcutaneous injection. Optionally, the vial 102 may contain multiple dosages, each of which is administered by a separate needle 132 and syringe 128.

In an alternative method, syringe 128 is produced containing the PT material 104 containing one or more chemical and/or biological species in the form of a dry powder. The needle 132 of syringe 128 is inserted into the vial 108 and plunger 130 pulled back to draw diluent 112 into the syringe 128. The incoming diluent 112 may dissolve the PT material. Optionally, the syringe 128 is shaken until the PT material 104 is essentially dissolved and the one or more chemical and/or biological species is thereby released. The reconstituted medicament (including PT material with one or more chemical and/or biological species) is then injected in step 134 into a patient.

In an alternative method, after the PT material 104 is dissolved, the vial 102 or its contents may be transferred to a nasal sprayer. For example, the cap 106 may be removed and a nasal sprayer head installed over the vial 102. For example, the nasal sprayer head may have a threaded or snap fit feature that engages a corresponding feature of the vial 102. The reconstituted medicament (i.e. an aqueous mixture of pullulan, trehalose and PT solution and one or more chemical and/or biological species, for example an antigen) may then be administered to a patient intranasally.

In some embodiments, diluent 112 is sterile water.

In some embodiments, a composition includes pullulan, trehalose, and one or more chemical and/or biological species, wherein the one or more chemical and/or biological species are incorporated within the polymer matrix and the polymer matrix preserves and/or stabilizes the chemical or biological species. In some embodiments, the composition is an immunogenic composition having a viral vector or an antigen. In some embodiments, the composition is essentially without or free from any other components, or at least without or free from any other components that are not generally regarded as safe (GRAS) and are suitable for administration to a person, for example by way of injection. In some embodiments, the composition is substantially without or free from surfactants. In some embodiments, the composition is without or free from PMAL-C16; without any, or without from about 0.1% to 10%, of a zwitterionic surfactant having a lipid group with a chain length of 13-30 carbon atoms; and/or, without any, or without from about 0.1 to 50 mg/ml, of a zwitterionic surfactant having a lipid group with a chain length of 13-30 carbon atoms. In some embodiments, the composition excludes any composition disclosed, for example by way of enabling disclosure and/or by way of specific example, in U.S. Pat. No. 9,974,850.

The following non-limiting examples are illustrative of the present application:

### Experimental

### Materials

Pullulan (PI20 food grade, 200 kDa) was obtained from Hayashibara Co, Ltd., Okayama, Japan. D-(+)-trehalose dehydrate, D-(+)-maltose monohydrate, sucrose, calcium chloride (CaCl₂)), magnesium sulfate (MgSO4.7H₂O), Tris, gelatin, Tryptic Soy Broth (TSB), and Listeria enrichment broth (LEB) were purchased from Sigma-Aldrich. Agar and agarose were purchased from Becton, Dickinson and Company (BD). Phosphate buffered saline (PBS) was purchased from BioShop Canada. Listeria monocytogenes serotype ½a, E. coli O157:H7, and Salmonella Newport, were routinely cultured and maintained in our lab. Two Myoviridae phages, E. coli O157:H7 phage, EcoM-AG10 (AG10), and Salmonella phage, SnpM-CG4-1 (CG4-1), were obtained from Canadian Research Institute for Food Safety, University of Guelph. LISTEX™ P100 was purchased from Micreos Food Safety (Wageningen, The Netherlands). Distilled deionized water was obtained from a Milli-Q Advantage A10 water purification system (EMD Millipore) and was autoclaved.

### Quantification of Infectivity for Phage Embedded in the Films

The infectivity of phages encapsulated in dried films was quantified using the overlay technique.⁸⁰ The phage-containing film was dissolved in 1 mL of CM buffer (prepared by mixing 2.5 g MgSO₄. 7H₂O, 0.735 g of CaCl₂, 0.05 g gelatin, and 6 mL 1 M Tris-HCl at pH 7.5, with water for a final volume of 1 L) through repeated pipetting. The reconstituted film solution was then serially diluted in CM buffer, each dilution was mixed in equal volumes with 100 μL of the bacterial host (10⁹ CFU/mL) and then incubated at 30° C. for 10 minutes to allow for phage adsorption. The phage-host mixture was then added to 4 mL of soft Tryptic Soy Agar (TSA, prepared by adding 0.5% agarose to TSB) and overlayed onto a TSA plate (1.5% agar to TSB). The plates were incubated at 30° C. overnight. Plaque formation was observed the following day, and the plaques were counted to determine the phage titer of each film. For each dilution, triplicate experiments were conducted. The total number of plaques were averaged and considered as the number of viable phages.

### Reconstitution and Powder Formation

An aqueous mixture, for example an injectable vaccine, may be reconstituted from a PT material including one or more chemical and/or biological species. In some cases, it is desirable to increase the rate of dissolution of the PT material. In some examples, this is achieved by preparing the PT material as a very thin film or in a powder form. For example, after drying a PT material can be converted to a powder through a mechanical process, such as milling, crushing or grinding a larger PT material.

An experiment was conducted to compare the rate of dissolution of an intact film to that of a film with a similar mass but crushed into smaller particles. The films were prepared as follows: from 10 mL solution containing 10% pullulan (0.1 g/mL=100 g/L) and 0.5 M trehalose, 90 uL was taken and mixed with 10 uL of a blue dye. Then, from this 100 uL mixture of pullulan/trehalose and dye, 60 uL was taken and dried for making PT films. The films were in the form of discs, about 5 mm in diameter and about 0.5 mm to 1 mm in thickness. Some samples of the PT film were left intact. Other samples of the PT film were manually crushed with a pipette tip. The PT films were brittle and crushing the PT film mostly produced a powder, but with several larger particles remaining that were not completely crushed to a powder form due to incomplete crushing. The larger particles were generally rectangular prisms with dimensions of about 0.5 to 1.5 mm on each side. Each of the larger particles had a volume of roughly 2-4% of the volume of the original film. The larger particles can be filtered out, but were left in the powder for the further procedures described below.

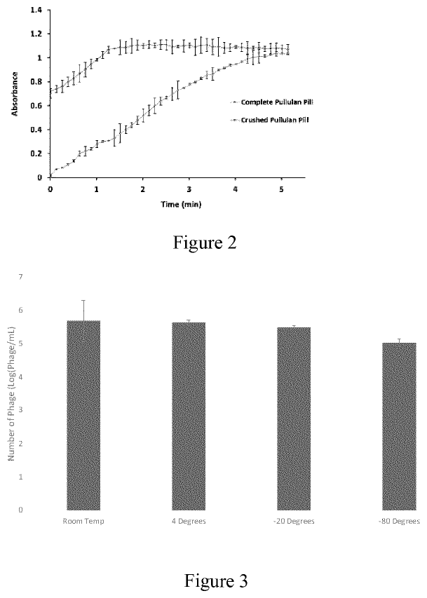

200 uL of water was added to various samples of intact film and crushed PT material. FIG. 2 shows the absorbance of dye released from the PT material over time starting from the addition of water at T=0. Increasing absorbance shows the rate of dissolution for the intact film and the crushed PT material. As indicated in FIG. 14, the rate of dissolution was increased, and the time to complete dissolution reduced by crushing the PT film. The results for the crushed PT material suggest that some of the smaller particles (i.e. the powdered portion of the PT material) dissolved almost instantaneously. However, the larger particles took some time (between 1-2 seconds in this example) to dissolve. The rate of dissolution may be related to the ratio of surface area to volume of a particle. This ratio increases as particle size decreases. In the context of a vaccine or other medicine that is reconstituted in the field, it is desirable for the PT material to be dissolve almost instantly after adding water, possibly with a few shakes or other mild agitation. Optionally, the PT material is produced such that particles of the PT material are produced having a surface area to volume ratio of 10-12:1 mm⁻¹ (i.e. the surface area to volume ratio of a 0.6 mm or 0.5 mm cube) or more.

To determine if powdering a PT material might have an impact on a biological species that is entrapped in the PT material, two PT films were created containing the phage P11 (useful against Pseudomonas aeruginosa). One PT film was maintained intact and the second PT film was physically disintegrated as described above. These two samples were then stored in a closed container for 2 days at room temperature. The number of viable phage was determined after dissolving each sample separately. The intact PT film yielded 5.67±0.04 log(phage/ml) of viable phage and the disintegrated PT material yielded 5.48±0.01 log(phage/ml) of viable phage. These results indicate that PT material can be physically powdered with very little loss of activity of a biological species preserved and/or stabilized by the PT material.

### Storage Under Varying Conditions

During transport or storage, it possible that PT films may be exposed to a wide range of temperatures. Experiments described above indicate that PT materials are effective at high temperatures, for example up to 40° C. However, shipping or storing the PT material during winter in or between cold countries may expose the PT material to low temperatures.

PT films were prepared containing the P11 phage and dried to about 7% moisture by weight. Samples of the films were stored for 4 days at room temperature, 4° C., −20° C. and −80° C. After this period of storage, the samples were dissolved and the number of viable phage counted. FIG. 3 shows the number of viable phage present in the PT films stored at the various temperatures. Storing the PT material at temperatures as low as −20° C. (5.5 log(phage/ml)) had very little impact on the number of viable phage relative to storage at room temperature (5.7 log(phage/ml)). Storage at −80° C. produced a more noticeable reduction in the number of viable phage (5.1 log(phage/ml)).

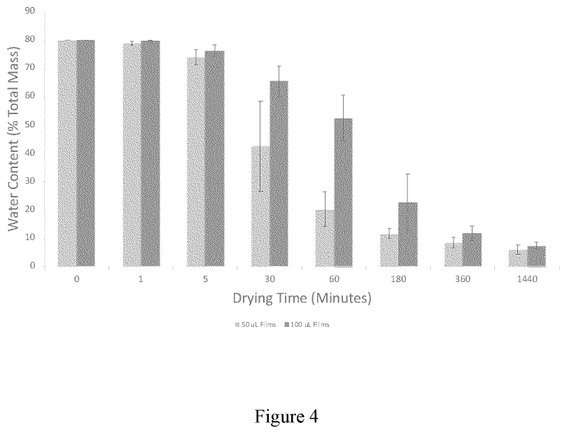

Previous work regarding the preparation and storage of PT films was described in Leung, V., L. Groves, A. Szewczyk, Z. Hosseinidoust, and C. D. Filipe, Long-Term Antimicrobial Activity of Phage-Sugar Glasses is Closely Tied to the Processing Conditions. ACS Omega, 2018. 3(12): p. 18295-18303, which is incorporated herein by reference. FIG. 3, panel B, of that publication shows that drying under vacuum produces less PFU reduction of the phage LISTEX P100 than drying in air. Although unreported at the time, the air had about 50% relative humidity (RH) and the moisture content of the PT films was over 10 wt % for the air-dried film and 6.0-7.3 wt % for the vacuum dried film. FIG. 4 of that publication shows that, after storage for 28 days, samples stored in open containers exposed to air at 58% relative humidity or more had higher PFU reduction of the phage LISTEX P100 than samples exposed to air at 33% relative humidity or less. Although unreported at the time, samples exposed to air with relative humidity of 33% and 11% had moisture contents of 10 wt % and 8 wt %. Collectively, this further analysis indicates that the PT material may be dried to, and maintained at, a moisture content of 10 wt % or less, or 8 wt % or less. It is optimal to store the PT material in a sealed container. The PT material may be filled and sealed into packages while operating in a suitably sterile environment having a relative humidity of up to 33-50%. Optionally, moisture in a package can be further reduced, for example by applying a vacuum to the packages before sealing them, since very low moisture content in the PT material appears to have no detrimental effect.

### Drying and Film Preparation

As mentioned above, the residual water content of the PT material may be kept at 10 wt % or less. Vacuum drying is more effective than drying with ambient air (RH typically of 50%) in reaching this amount of residual water. FIG. 4 shows in greater detail how the residual water content of solutions of PT material with different volumes (50 uL and 100 uL) changes as a function of time under vacuum drying. The larger volume requires a longer time to achieve residual water content of less than 10 wt %: 6 hours for the 50 uL sample and between 6-24 hours for the 100 uL sample. At 24 hours, the residual water content was 6 wt % and 7.3 wt % for 50 uL and 100 uL samples, respectively.

When samples of these volumes (50 uL and 100 uL) are placed in a vessel large enough for the solution to form a droplet the sample dries to a thin (1 mm or less) film covering approximately the footprint of the original droplet. For example, a 50 uL droplet produces a 5-7 mm diameter film. A 100 uL droplet produces a film of about 10 mm in diameter.

Drying should be done such that the formation of crystals is avoided. Small particles in the solution, or formation of crystals through the presence of certain salts (such as phosphate), may lead to loss of the protective ability of the PT material.

No negative effects have been noticed with rapid drying, even to the extent of drying in 2 minutes produced by applying a nitrogen jet to a thin liquid film of PT material. On the contrary, faster drying can reduce degradation of chemical or biological species that degrade in aqueous environments. In some cases, rapid drying under vacuum can cause bubbling in the PT material as it dries. This may modify the shape or form of the dried PT material, but does not appear to reduce the protective ability of the PT material.

Methods that create high shear conditions during the preparation of PT materials can reduce the stability of at least some biological species. In one experiment, an aqueous mixture of pullulan, trehalose and P11 phage was split in two aliquots. The first aliquot was used to create PT films without using vortexing to mix the phage with the pullulan/trehalose solution and the second aliquot was subjected to 60 seconds of vortexing. These two films were reconstituted after 4 days of storage at room temperature. The sample that was not vortexed yielded 5.67±0.04 log(phage/ml) and the sample that was subjected to vortexing yielded 4.2±0.05 log(phage/ml). In this case, vortexing resulted in a substantial loss of phage counts.

### Effect of Method of Drying on Film Formation

Pullulan/trehalose films using three different drying methods: (1) through vacuum drying; (2) through freeze drying (prior art method) and (3) through spray drying. For all the cases, the initial solution contained 10% pullulan and 0.5 M trehalose. The resulting solid films obtained using the three different methods of drying, were observed through optical polarized light microscopy (transmission and/or reflective mode). This microscopic technique is a widely used method to detect the presence of crystals in samples. The crystals can be identified by the presence of structures that have a rainbow-like glow associated with them. The solids obtained using vacuum drying, did not contain any crystals, whereas the solids obtained either through freeze-drying or through spray drying, contained crystals.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATION FOR DOCUMENTS REFERRED TO IN THE SPECIFICATION

(1) Smith, H. W.; Huggins, M. B. Successful Treatment of Experimental Escherichia coli Infections in Mice Using Phage: its General Superiority over Antibiotics. Microbiology 1982, 128 (2), 307-318 DOI: 10.1099/00221287-128-2-307.
(2) Kutateladze, M. Experience of the Eliava Institute in bacteriophage therapy. Virol. Sin. 2015, 30 (1), 80-81 DOI: 10.1007/s12250-014-3557-0.
(3) Ye, J.; Kostrzynska, M.; Dunfield, K.; Warriner, K. Evaluation of a biocontrol preparation consisting of Enterobacter asburiae JX1 and a lytic bacteriophage cocktail to suppress the growth of Salmonella Javiana associated with tomatoes. J. Food Prot. 2009, 72 (11), 2284-2292.
(4) Sharma, M.; Patel, J. R.; Conway, W. S.; Ferguson, S.; Sulakvelidze, A. Effectiveness of bacteriophages in reducing Escherichia coli O157:H7 on fresh-cut cantaloupes and lettucet. J. Food Prot. 2009, 72 (7), 1481-1485.
(5) Abuladze, T.; Li, M.; Menetrez, M. Y.; Dean, T.; Senecal, A.; Sulakvelidze, A. Bacteriophages reduce experimental contamination of hard surfaces, tomato, spinach, broccoli, and ground beef by Escherichia coli O157:H7. Appl. Environ. Microbiol. 2008, 74 (20), 6230-6238 DOI: 10.1128/AEM.01465-08.
(6) Spricigo, D. A.; Bardina, C.; Cortés, P.; Llagostera, M. Use of a bacteriophage cocktail to control Salmonella in food and the food industry. Int. J. Food Microbiol. 2013, 165 (2), 169-174 DOI: 10.1016/j.ijfoodmicro.2013.05.009.
(7) Anany, H.; Chen, W.; Pelton, R.; Griffiths, M. W. Biocontrol of Listeria monocytogenes and Escherichia coli O157:H7 in meat by using phages immobilized on modified cellulose membranes. Appl. Environ. Microbiol. 2011, 77 (18), 6379-6387 DOI: 10.1128/AEM. 05493-11.
(8) Lone, A.; Anany, H.; Hakeem, M.; Aguis, L.; Avdjian, A.-C.; Bouget, M.; Atashi, A.; Brovko, L.; Rochefort, D.; Griffiths, M. W. Development of prototypes of bioactive packaging materials based on immobilized bacteriophages for control of growth of bacterial pathogens in foods. Int. J. Food Microbiol. 2016, 217, 49-58 DOI: 10.1016/j.ijfoodmicro.2015.10.011.
(9) Seo, J.; Seo, D. J.; Oh, H.; Jeon, S. B.; Oh, M.; Choi, C. Inhibiting the Growth of Escherichia coli O157: H7 in Beef, Pork, and Chicken Meat using a Bacteriophage. 2016, 36 (2), 186-193 DOI: 10.5851/kosfa.2016.36.2.186.
(10) Carocho, M.; Morales, P.; Ferreira, I. C. F. R. Natural food additives: Quo vadis? Trends Food Sci. Technol. 2015, 45 (2), 284-295 DOI: 10.1016/j.tifs.2015.06.007.
(11) Janež N.; Loc-Carrillo, C. Use of phages to control campylobacter spp. J. Microbiol. Methods 2013, 95 (1), 68-75 DOI: 10.1016/j.mimet.2013.06.024.
(12) Hudson, J. A.; Billington, C.; Carey-Smith, G.; Greening, G. Bacteriophages as biocontrol agents in food. J. Food Prot. 2005, 68 (2), 426-437.
(13) Mahony, J.; McAuliffe, 0.; Ross, R. P.; van Sinderen, D. Bacteriophages as biocontrol agents of food pathogens. Curr. Opin. Biotechnol. 2011, 22 (2), 157-163 DOI: 10.1016/j.copbio.2010.10.008.
(14) Cademartiri, R.; Anany, H.; Gross, I.; Bhayani, R.; Griffiths, M.; Brook, M. a. Immobilization of bacteriophages on modified silica particles. Biomaterials 2010, 31 (7), 1904-1910 DOI: 10.1016/j.biomaterials.2009.11.029.
(15) Choińska-Pulit, A.; Mitula, P.; Śliwka, P.; Laba, W.; Skaradzińiska, A. Bacteriophage encapsulation: Trends and potential applications. Trends Food Sci. Technol. 2015, 45 (2), 212-221 DOI: 10.1016/j.tifs.2015.07.001.
(16) Vonasek, E.; Le, P.; Nitin, N. Encapsulation of bacteriophages in whey protein films for extended storage and release. Food Hydrocoll. 2014, 37, 7-13 DOI: 10.1016/j.foodhyd.2013.09.017.
(17) Shea, K. M. Antibiotic Resistance: What Is the Impact of Agricultural Uses of Antibiotics on Children's Health? Pediatrics 2003, 112 (1), 253-258 DOI: 10.1542/peds.112.1. S1.253.
(18) Lau, C. H.-F.; van Engelen, K.; Gordon, S.; Renaud, J.; Topp, E. Novel Antibiotic Resistance Determinants from Agriculturual Soil Exposed to Antibiotics Widely Used in Human Medicine and Animal Farming. Appl. Environ. Microbiol. 2017, No. June, AEM.00989-17 DOI: 10.1128/AEM.00989-17.
(19) Azeredo, J.; Sutherland, I. The Use of Phages for the Removal of Infectious Biofilms. Curr. Pharm. Biotechnol. 2008, 9 (4), 261-266 DOI: 10.2174/138920108785161604.
(20) Jonczyk, E.; Klak, M.; Międzybrodzki, R.; Górski, A. The influence of external factors on bacteriophages-review. Folia Microbiol. (Praha). 2011, 56 (3), 191-200 DOI: 10.1007/s12223-011-0039-8.
(21) Puapermpoonsiri, U.; Spencer, J.; van der Walle, C. F. A freeze-dried formulation of bacteriophage encapsulated in biodegradable microspheres. Eur. J. Pharm. Biopharm. 2009, 72 (1), 26-33 DOI: 10.1016/j.ejpb.2008.12.001.
(22) Puapermpoonsiri, U.; Ford, S. J.; van der Walle, C. F. Stabilization of bacteriophage during freeze drying. Int. J. Pharm. 2010, 389 (1-2), 168-175 DOI: 10.1016/j.ijpharm.2010.01.034.
(23) Balcão, V. M.; Barreira, S. V. P.; Nunes, T. M.; Chaud, M. V.; Tubino, M.; Vila, M. M. D. C. Carbohydrate Hydrogels with Stabilized Phage Particles for Bacterial Biosensing: Bacterium Diffusion Studies. Appl. Biochem. Biotechnol. 2014, 172 (3), 1194-1214 DOI: 10.1007/s 12010-013-0579-2.
(24) Dini, C.; Islan, G. A.; de Urraza, P. J.; Castro, G. R. Novel biopolymer matrices for microencapsulation of phages: Enhanced protection against acidity and protease activity. Macromol. Biosci. 2012, 12 (9), 1200-1208 DOI: 10.1002/mabi.201200109.
(25) Moghtader, F.; E\check{g}ri, S.; Piskin, E. Phages in modified alginate beads. Artif Cells, Nanomedicine, Biotechnol. 2017, 45 (2), 357-363 DOI: 10.3109/21691401.2016.1153485.
(26) Kim, S.; Jo, A.; Ahn, J. Application of chitosan-alginate microspheres for the sustained release of bacteriophage in simulated gastrointestinal conditions. Int. J. Food Sci. Technol. 2015, 50 (4), 913-918 DOI: 10.1111/ijfs.12736.
(27) Colom, J.; Cano-Sarabia, M.; Otero, J.; Ariñez-Soriano, J.; Cortés, P.; Maspoch, D.; Llagostera, M. Microencapsulation with alginate/CaCO3: A strategy for improved phage therapy. Sci. Rep. 2017, 7 (December 2016), 41441 DOI: 10.1038/srep41441.
(28) Ma, Y.; Pacan, J. C.; Wang, Q.; Sabour, P. M.; Huang, X.; Xu, Y. Enhanced alginate microspheres as means of oral delivery of bacteriophage for reducing Staphylococcus aureus intestinal carriage. Food Hydrocoll. 2012, 26 (2), 434-440 DOI: 10.1016/j.foodhyd.2010.11.017.
(29) Dini, C.; Islan, G. A.; Castro, G. R. Characterization and Stability Analysis of Biopolymeric Matrices Designed for Phage-Controlled Release. Appl. Biochem. Biotechnol. 2014, 174 (6), 2031-2047 DOI: 10.1007/s12010-014-1152-3.
(30) Tang, Z.; Huang, X.; Baxi, S.; Chambers, J. R.; Sabour, P. M.; Wang, Q. Whey protein improves survival and release characteristics of bacteriophage Felix O1 encapsulated in alginate microspheres. Food Res. Int. 2013, 52 (2), 460-466 DOI: 10.1016/j.foodres.2012.12.037.
(31) Colom, J.; Cano-Sarabia, M.; Otero, J.; Cort??s, P.; Maspoch, D.; Llagostera, M. Liposome-encapsulated bacteriophages for enhanced oral phage therapy against Salmonella spp. Appl. Environ. Microbiol. 2015, 81 (14), 4841-4849 DOI: 10.1128/AEM.00812-15.
(32) Singla, S.; Harjai, K.; Katare, 0. P.; Chhibber, S. Encapsulation of bacteriophage in liposome accentuates its entry in to macrophage and shields it from neutralizing antibodies. PLoS One 2016, 11 (4), 1-16 DOI: 10.1371/journal.pone.0153777.
(33) Korehei, R.; Kadla, J. F. Encapsulation of T4 bacteriophage in electrospun poly(ethylene oxide)/cellulose diacetate fibers. Carbohydr. Polym. 2014, 100, 150-157 DOI: 10.1016/j.carbpol.2013.03.079.
(34) Merabishvili, M.; Vervaet, C.; Pirnay, J.; Vos, D. De; Verbeken, G.; Mast, J.; Chanishvili, N.; Vaneechoutte, M. Stability of Staphylococcus aureus Phage ISP after Freeze-Drying (Lyophilization). PLoS One 2013, 8 (7), 1-7 DOI: 10.1371/j ournal.pone.0068797.
(35) Matthias, D. M., Robertson, J., Garrison, M. M., Newland, S. & Nelson, C. Freezing temperatures in the vaccine cold chain: A systematic literature review. Vaccine 25, 3980-3986 (2007).
(36) Levin, A., Levin, C., Kristensen, D. & Matthias, D. An economic evaluation of thermostable vaccines in Cambodia, Ghana and Bangladesh. Vaccine 25, 6945-6957 (2007).
(37) Favin, M., Steinglass, R., Fields, R., Banerjee, K. & Sawhney, M. Why children are not vaccinated: A review of the grey literature. Int. Health 4, 229-238 (2012).
(38) Luzze, H. et al. Understanding the policy environment for immunization supply chains: Lessons learned from landscape analyses in Uganda and Senegal. Vaccine 35, 2141-2147 (2017).
(39) Azimi, T., Franzel, L. & Probst, N. Seizing market shaping opportunities for vaccine cold chain equipment. Vaccine 35, 2260-2264 (2017).
(40) Sun, T. et al. Thermal stability of self-assembled peptide vaccine materials. Acta Biomater. 30, 62-71 (2016).
(41) Konar, M., Pajon, R. & Beernink, P. T. A meningococcal vaccine antigen engineered to increase thermal stability and stabilize protective epitopes. Proc. Natl. Acad. Sci. 112, 14823-14828 (2015).
(42) Rossi, R., Konar, M. & Beernink, P. T. Meningococcal factor H binding protein vaccine antigens with increased thermal stability and decreased binding of human factor H. Infect. Immun. 84, 1735-1742 (2016).
(43) Campeotto, I. et al. One-step design of a stable variant of the malaria invasion protein RH5 for use as a vaccine immunogen. Proc. Natl. Acad. Sci. 114, 998-1002 (2017).
(44) Stobart, C. C. et al. A live RSV vaccine with engineered thermostability is immunogenic in cotton rats despite high attenuation. Nat. Commun. 7, 1-12 (2016).
(45) Wang, G. et al. Rational design of thermostable vaccines by engineered peptide-induced virus self-biomineralization under physiological conditions. 1-6 (2013). doi:10.1073/pnas.1300233110
(46) Pelliccia, M. et al. Additives for vaccine storage to improve thermal stability of adenoviruses from hours to months. Nat. Commun. 7, 1-7 (2016).
(47) Chu, L. Y. et al. Enhanced Stability of Inactivated Influenza Vaccine Encapsulated in Dissolving Microneedle Patches. Pharm. Res. 33, 868-878 (2016).
(48) Choi, H.-J. et al. Stability of influenza vaccine coated onto microneedles. Biomaterials 33, 3756-3769 (2012).
(49) Mistilis, M. J. et al. Long-term stability of influenza vaccine in a dissolving microneedle patch. Drug Deliv. Transl. Res. 7, 195-205 (2017).
(50) Hassett, K. J. et al. Glassy-State Stabilization of a Dominant Negative Inhibitor Anthrax Vaccine Containing Aluminum Hydroxide and Glycopyranoside Lipid A Adjuvants. J. Pharm. Sci. 104, 627-639 (2015).
(51) Hassett, K. J. K. et al. Stabilization of a recombinant ricin toxin A subunit vaccine through lyophilization. Eur. J. Pharm. Biopharm. 85, 279-86 (2013).
(52) Chen, D. et al. Thermostable formulations of a hepatitis B vaccine and a meningitis A polysaccharide conjugate vaccine produced by a spray drying method. Vaccine 28, 5093-5099 (2010).
(53) Ohtake, S. et al. Room temperature stabilization of oral, live attenuated Salmonella enterica serovar Typhi-vectored vaccines. Vaccine 29, 2761-71 (2011).
(54) Ohtake, S. et al. Heat-stable measles vaccine produced by spray drying. Vaccine 28, 1275-84 (2010).
(55) Lovalenti, P. M. et al. Stabilization of live attenuated influenza vaccines by freeze drying, spray drying, and foam drying. Pharm. Res. 33, 1144-1160 (2016).
(56) Madan, M. et al. Rational design of heat stable lyophilized rotavirus vaccine formulations. Hum. Vaccin. Immunother. 5515, 1-10 (2018).
(57) Naik, S. P. et al. Stability of heat stable, live attenuated Rotavirus vaccine (ROTASIIL®). Vaccine 35, 2962-2969 (2017).
(58) Alcock, R. et al. Long-Term Thermostabilization of Live Poxviral and Adenoviral Vaccine Vectors at Supraphysiological Temperatures in Carbohydrate Glass. Sci. Transl. Med. 2, 19ra12-19ra12 (2010)
(59) Wu, S.; Chen, J. Using pullulan-based edible coatings to extend shelf-life of fresh-cut “Fuji” apples. Int. J. Biol. Macromol. 2013, 55, 254-257 DOI: 10.1016/j.ijbiomac.2013.01.012.
(60) Farris, S.; Introzzi, L.; Fuentes-Alventosa, J. M.; Santo, N.; Rocca, R.; Piergiovanni, L. Self-Assembled Pullulan-Silica Oxygen Barrier Hybrid Coatings for Food Packaging Applications. J. Agric. Food Chem. 2012, 60 (3), 782-790 DOI: 10.1021/jf204033d.
(61) Wu, S. & Chen, J. Using pullulan-based edible coatings to extend shelf-life of fresh-cut ‘Fuji’ apples. Int. J. Biol. Macromol. 55, 254-257 (2013).
(62) Treviño-Garza, M. Z., Garcia, S., del Socorro Flores-Gonzalez, M. & Arévalo-Niño, K. Edible Active Coatings Based on Pectin, Pullulan, and Chitosan Increase Quality and Shelf Life of Strawberries (Fragaria ananassa). J. Food Sci. 80, M1823-M1830 (2015).
(63) Kraśniewska, K. et al. Effect of Pullulan Coating on Postharvest Quality and Shelf-Life of Highbush Blueberry (Vaccinium corymbosum L.). Materials (Basel). 10, 965 (2017).
(64) Morsy, M. K., Sharoba, A. M., Khalaf, H. H., El-Tanahy, H. H. & Cutter, C. N. Efficacy of Antimicrobial Pullulan-Based Coating to Improve Internal Quality and Shelf-Life of Chicken Eggs During Storage. J. Food Sci. 80, M1066-M1074 (2015).
(65) Farris, S. et al. Self-Assembled Pullulan-Silica Oxygen Barrier Hybrid Coatings for Food Packaging Applications. J. Agric. Food Chem. 60, 782-790 (2012).
(66) Jahanshahi-Anbuhi, S.; Kannan, B.; Leung, V.; Pennings, K.; Liu, M.; Carrasquilla, C.; White, D.; Li, Y.; Pelton, R. H.; Brennan, J. D.; et al. Simple and ultrastable all-inclusive pullulan tablets for challenging bioassays. Chem. Sci. 2016, 7, 2342-2346 DOI: 10.1039/C5SC04184H.
(67) Jahanshahi-Anbuhi, S.; Pennings, K.; Leung, V.; Liu, M.; Carrasquilla, C.; Kannan, B.; Li, Y.; Pelton, R.; Brennan, J. D.; Filipe, C. D. M. Pullulan Encapsulation of Labile Biomolecules to Give Stable Bioassay Tablets. Angew. Chemie Int. Ed. 2014, 53 (24), 6155-6158 DOI: 10.1002/anie.201403222.
(68) Jain, N. K.; Roy, I. Effect of trehalose on protein structure. Protein Sci. 2008, No. September 2008, 24-36 DOI: 10.1002/pro.3.
(69) Tapia, H.; Koshland, D. E. Trehalose Is a Versatile and Long-Lived Chaperone for Desiccation Tolerance. Curr. Biol. 2014, 24, 2758-2766 DOI: 10.1016/j.cub.2014.10.005.
(70) Iyer, P. V.; Ananthanarayan, L. Enzyme stability and stabilization-Aqueous and non-aqueous environment. Process Biochem. 2008, 43 (10), 1019-1032 DOI: 10.1016/j.procbio.2008.06.004.
(71) Ohtake, S.; Wang, Y. J. Trehalose: Current use and future applications. J. Pharm. Sci. 2011, 100 (6), 2020-2053 DOI: 10.1002/jps.22458.
(72) Vandenheuvel, D.; Meeus, J.; Lavigne, R.; Van Den Mooter, G. Instability of bacteriophages in spray-dried trehalose powders is caused by crystallization of the matrix. Int. J. Pharm. 2014, 472 (1-2), 202-205 DOI: 10.1016/j.ijpharm.2014.06.026.
(73) Vandenheuvel, D.; Singh, A.; Vandersteegen, K.; Klumpp, J.; Lavigne, R.; Van Den Mooter, G. Feasibility of spray drying bacteriophages into respirable powders to combat pulmonary bacterial infections. Eur. J. Pharm. Biopharm. 2013, 84 (3), 578-582 DOI: 10.1016/j.ejpb.2012.12.022.
(74) Jain, N. K. & Roy, I. Effect of trehalose on protein structure. Protein Sci. 24-36 (2008). doi:10.1002/pro.3
(75) Tapia, H. & Koshland, D. E. Trehalose Is a Versatile and Long-Lived Chaperone for Desiccation Tolerance. Curr. Biol. 24, 2758-2766 (2014).
(76) Iyer, P. V. & Ananthanarayan, L. Enzyme stability and stabilization-Aqueous and non-aqueous environment. Process Biochem. 43, 1019-1032 (2008).
(77) Ohtake, S. & Wang, Y. J. Trehalose: Current use and future applications. J. Pharm. Sci. 100, 2020-2053 (2011).
(78) Vandenheuvel, D., Meeus, J., Lavigne, R. & Van Den Mooter, G. Instability of bacteriophages in spray-dried trehalose powders is caused by crystallization of the matrix. Int. J. Pharm. 472, 202-205 (2014).
(79) Vandenheuvel, D. et al. Feasibility of spray drying bacteriophages into respirable powders to combat pulmonary bacterial infections. Eur. J. Pharm. Biopharm. 84, 578-582 (2013).
(80) Kropinski, A. M.; Mazzocco, A.; Waddell, T. E.; Lingohr, E.; Johnson, R. P. Enumeration of Bacteriophages by Double Agar Overlay Plaque Assay; 2009; pp 69-76.

1. A polymer matrix comprising pullulan and trehalose and one or more chemical and/or biological species, wherein the one or more chemical and/or biological species are incorporated within the polymer matrix and the polymer matrix preserves and/or stabilizes the one or more chemical and/or biological species, and the polymer matrix is in powder form.

2. The polymer matrix of claim 1 comprising about 10 wt % to about 50 wt % of pullulan and about 50 wt % to about 90 wt % of trehalose, based on the dry weight of the matrix.

3. The polymer matrix of claim 1, wherein the pullulan has a molecular weight in the range of about 100,000 to about 200,000.

4. The polymer matrix of claim 1, wherein the one or more chemical and/or biological species are preserved at a temperature of from about −20° C. to about 40° C.

5. The polymer matrix of claim 4, wherein the one or more chemical and/or biological species are preserved and/or stabilized for at least 4 days at the temperatures below freezing.

6. The polymer matrix of claim 1, wherein the one or more chemical species is a biomolecule is chosen from one or more of a protein, an enzyme, an antibody, a peptide, a nucleic acid, an antidote and a vaccine.

7. The polymer matrix of claim 1, wherein the one or more chemical species are biomolecules that act as immunogens or that are used to generate an immune response.

8. The polymer matrix of claim 1, wherein the one or more biological species is a microorganism chosen from one or more of anaerobic bacteria, aerobic bacteria, mammalian cells, bacterial cells and viruses.

9. The polymer matrix of claim 8, wherein the microorganism is a virus.

10. The polymer matrix of claim 1, wherein the polymer matrix further comprises one or more additional substances or additives.

11. The polymer matrix of claim 1, wherein the polymer matrix is essentially free from any additives that are non-GRAS or not acceptable for injection in a person, the polymer matrix is essentially free from PMAL-C16, the polymer matrix is essentially free from, or contains less than 0.1% to 10%, of a zwitterionic surfactant having a lipid group with a chain length of 13-30 carbon atoms, or the polymer matrix is free from, or contains less than 0.1 mg/ml to 50 mg/ml, of a zwitterionic surfactant having a lipid group with a chain length of 13-30 carbon atoms.

12. The polymer matrix of claim 1, wherein the polymer matrix is essentially free from additives.

13. The polymer matrix of claim 1, wherein the powder comprises particles having a median surface area:volume ratio of 10:1 mm⁻¹ to about 120:1 mm⁻¹.

14. The polymer matrix of claim 1, having a water content of less than 10 wt %.

15. A method of preserving and/or stabilizing one or more chemical and/or biological species comprising:
a. combining the one or more chemical and/or biological species, pullulan and trehalose and water to provide a mixture;
b. drying the mixture to form a polymer matrix which preserves and/or stabilizes the one or more chemical and/or biological species; and
c. converting the polymer matrix into powder form.

16-22. (canceled Herein)

23. A method of providing an immune response in a mammal comprising administering an effective amount of the polymer matrix of claim 1, to a mammal in need thereof.

24. The method of claim 23 comprising adding a diluent to the powder to provide an aqueous mixture and administering the aqueous mixture to the mammal.

25. An immunogenic composition comprising an antigen, the antigen formulated in a polymer matrix of claim 1.

26-27. (canceled Herein)

28. A method of making and/or storing and/or transporting a stabilized medicament or component thereof comprising a) producing a mixture of pullulan, trehalose and one or more chemical and/or biological species, b) drying the mixture to 10 wt % moisture content or below to provide a dried material and c) packaging the dried material in a sealed container.

29-32. (canceled Herein)

33. The method of claim 23, wherein the aqueous mixture is administered by injecting the aqueous mixture into the mammal or by an intranasal route by way of an atomizer or sprayer.
